(19) United States
(12) Patent Application Publication
(10) Pub. No.: US 2007/0259859 A1
(43) Pub. Date: Nov. 8, 2007

(54) MUSCARINIC AGONISTS TO TREAT IMPULSE CONTROL DISORDERS

(76) Inventors: Natasja M.W.J. De Bruin, Weesp (NL); Marlies L. Van Drimmelen, Weesp (NL); Arnoldus H.J. Herremans, Weesp (NL); Martinus Th.M. Tulp, Weesp (NL); Cornelis G. Kruse, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/741,976

(22) Filed: Apr. 30, 2007

Related U.S. Application Data
(60) Provisional application No. 60/797,355, filed on May 4, 2006.

(51) Int. Cl. A61K 31/5513

(52) U.S. Cl. 514/221

(57) ABSTRACT

The invention relates to a novel use of compounds and pharmaceutically acceptable salts thereof, which are muscarinic, for example muscarine-1 (M₁) agonists. These compounds are useful for the preparation of medicaments for treatment, amelioration or prevention of impulse control disorders. These include impulse control disorders ‘Not Elsewhere Classified,’ such as intermittent explosive disorder, pyromania, kleptomania, pathological gambling and trichotillomania, and impulse control disorders ‘Not Otherwise Specified,’ such as compulsive buying disorder, binge eating and binge drinking disorder, impulsive self-injurious behavior, sexual addictions, compulsive Internet use, and excessive mobile phone use.

This application claims the benefit of U.S. provisional Application No. 60/797,355, filed May 4, 2006, the entirety of which is incorporated herein by reference.

The present disclosure is generally directed to the novel use of muscarinic agonists, and their salts, for the treatment, amelioration or prevention of impulse control disorders. An impulsive person is apt to be moved by a sudden impulse. This behavior often is associated with a lack of self-control.

Impulsivity therefore, has a substantial impact on individuals as well as on society. Impulse control disorders (ICD's) are characterized by the failure to resist an impulse, drive or temptation to perform an act that is harmful to the person or to others. In most cases, the individual feels an increasing sense of tension or arousal before committing the act, and then experiences pleasure, gratification, or release at the time of committing the act. After the act is performed, there may or may not be regret or guilt.

Impulse control disorders are a separate group of psychiatric disorders, listed in the “Diagnostic and Statistical Manual of Mental Disorders” (DSM-IV) of the American Psychiatric Association as a residual category consisting of impulse control disorders ‘Not Elsewhere Classified’ (NEC) and impulse control disorders ‘Not Otherwise Specified’ (NOS). The first includes: intermittent explosive disorder, pyromania, kleptomania, pathological gambling and trichotillomania. No specific disorders are mentioned in DSM-IV under the heading: ‘impulse control disorders NOS’, but this group is defined as “a category for disorders of impulse control that do not meet the criteria for any specific impulse control disorder or for any other mental disorder having features involving impulse control (such as borderline, antisocial, histrionic and narcissistic personality disorders)”. In the scientific and patent literature a number of such impulse control disorders, also referred to as ‘atypical impulse control disorders’, are described, for instance: compulsive buying disorder, binge eating and binge drinking disorders, impulsive self-injurious behavior, such as pathological skin picking, nail-biting and nose-picking, gouging, head banging and self-biting, paraphilic sexual addictions, lack of control of a person's sexual impulses, including exhibitionism, fetishism, frotteurism, pedophilia, masochism, sadism, transvestic fetishism and voyeurism, as well as compulsive Internet use and excessive mobile phone use.

Patients suffering from an impulse control disorder have to date been treated by psychotherapy, behavior modification, hypnosis, relaxation therapy and administration of varied pharmaceutical preparations, the latter with little or no success. Historically, impulse control disorders have been considered refractory to known pharmacological or psychotherapeutic treatments. Therefore, a continuing need exists for agents that will be effective to treat the symptoms associated with ICD's, either by eliminating or by reducing them.

In different patent and patent applications a variety of molecular mechanisms are claimed to be of therapeutic value in impulse control disorders: opioid antagonists (U.S. Pat. No. 5,780,479), anticonvulsants (WO 02/43731); serotonin antagonists (US 2001023254); 5-HTlA agonists (WO 94/13659), serotonin reuptake inhibitors (WO 92/18005) and cannabinoid antagonists (US 2004/0077650). Muscarinic agonists have been disclosed for treatment for cognitive impairment, psychosis, affective disorders, mania, and behavioral disorders (WO 2006/067494, WO 2006/017614 and EP 0525 879), as well as for the treatment of tics, tremors and related disorders (US 2004/116505). These disorders are quite distinct from impulse control disorders (DSM-IV).

### DETAILED DESCRIPTION OF THE INVENTION

A goal of the present invention is to develop one or more drugs for the therapy of impulse control disorders, which have a mechanism of action different from that of drugs currently on the market, and which are likely to improve impulsivity control without having negative effects on attention and concentration.

Surprisingly, muscarinic agonists were found active in an animal model predictive of impulsive behavior in humans: the attenuation of MK801 induced increase of anticipatory responses of rats in the 5-Choice-Serial-Reaction-Time task, an effect associated with impulsive behavior (Cole, 1987; Ruotsalainen, 2000). Muscarinic agonists are active at doses in the range of 0.1-100 mg/kg after oral administration, and their unique pharmacological profile clearly indicates therapeutic potential in impulse control disorders.

In one embodiment, the invention embraces one or more of the following muscarinic agonists: AF-150, AF-151, alvameline, ACP-104, CDD-34, CDD-98, CDD-0097, CDD-0102, CDD-190, CDD-0199-J, CDD-0235-J, CDD-0304, cevimeline, CPR-2006, CS-932, desmethyliclozapine, FPL-14995, FPL-15467, KAD-193R, L-680648, L-687306, L-689660, MCNa-343, milameline, nebracetam, NGX-267, PD-151832, sabcomeline, SDZ-210-086, SR-46559A, talsaclidine fumarate, tazomeline, xanomeline, YM-796 and YM-954.

Use of the muscarinic agonists xanomeline and desmethylclozapine are contemplated.

The compounds of the invention of the general formula (1), as well as the pharmacologically acceptable salts thereof, have muscarinic receptor agonistic activity. They are useful in treating impulse control disorders, including intermittent explosive disorder, pyromania, kleptomania, pathological gambling, trichotillomania, compulsive buying disorder, binge eating and binge drinking disorder; impulsive self-injurious behavior such as pathological skin picking, nail-biting, nose-picking, gouging, head banging and self-biting; paraphilic sexual addictions, including exhibitionism, fetishism, frotteurism, pedophilia, masochism, sadism, transvestic fetishism and voyeurism; compulsive Internet use and excessive mobile phone use.

From the DSM-IV: Impulse Control Disorders Not Elsewhere Classified (NEC):

Intermittent explosive disorder (IED) is a disease characterized by the occurrence of discrete episodes of failure to resist aggressive impulses that result in serious assaultive acts or destruction of property. The degree of aggressiveness expressed during an episode is grossly out of proportion to any provocation or precipitating psychosocial stressor. The diagnosis IED is made only after other mental disorders that might account for episodes of aggressive behavior have been ruled out (e.g., antisocial personality disorder, borderline personality disorder, a psychotic disorder, a manic episode, conduct disorder or ADHD. In IED, the aggressive episodes are not due to the direct physiological effects of a substance (e.g., an abused drug or medication) or a general medical condition (e.g., head trauma). Patients may describe the aggressive episodes as ‘spells’ or ‘attacks’ in which the explosive behavior is preceded by a sense of tension or arousal and is followed immediately by a sense of relief. Later the individual may feel upset, remorseful, regretful or embarrassed about the aggressive behavior.

Pyromania, also referred to as arsonism, pyrophilia or pathological fire-setting, is characterized by the presence of multiple episodes of deliberate and purposeful fire setting. Patients experience tension or affective arousal before setting a fire, and are characterized by fascination with, interest in, curiosity about, or attraction to fire and its situational contexts. Individuals with this disorder are often regular watchers at fires in their neighborhoods, may set off false alarms, and derive pleasure from institutions, equipment and personnel associated with fire. Pyromaniacs experience pleasure, gratification of a release of tension when setting a fire, witnessing its effects or participating in its aftermath. The fire setting is not done for monetary gain, as an expression of sociopolitical ideology, to conceal criminal activity, to express anger or vengeance, to improve one's living circumstances, or in response to a delusion or a hallucination, and the fire setting does not result from impaired judgement (e.g., dementia, mental retardation or substance abuse).

Kleptomania is characterized by a recurrent failure to resist impulses to steal items even though they are not needed for personal use or for their monetary value. The individual experiences a rising subjective sense of tension before the theft and feels pleasure, gratification or relief when committing the theft. The stealing is not committed to express anger or vengeance, is not done in response to a delusion or hallucination, and is not better counted for by conduct disorder, a manic episode or antisocial personality disorder. The objects are stolen despite the fact that are typically of little value to the individual who could have afforded to pay for them, and often gives them away or discards them. Although patients will generally avoid stealing when immediate arrest is probable, they usually do not preplan the thefts or fully take into account the chances of apprehension. The stealing is done without assistance from, or collaboration with, others.

Pathological gambling, also known as ‘gambling disorder’ or ‘problem gambling,’ is characterized by a persistent and recurrent maladaptive gambling behavior that disrupts personal, family or vocational pursuits. The individual may be preoccupied with gambling. Most patients say that they are seeking ‘action’ even more than money. Increasingly larger bets, or greater risks, may be needed to continue to produce the desired level of excitement. Gambling often continues despite repeated efforts to control, cut back, or stop the behavior. The individual may gamble as a way of escaping from problems or to relieve a dysphoric mood. A pattern of ‘chasing’ one's losses may develop, with an urgent need to keep gambling to undo losses. Individuals may lie to family members, therapists or others to conceal the extend of involvement with gambling. When his borrowing resources are restrained, the person may resort to antisocial behavior (fraud, theft) to obtain money. The individual may have jeopardized or lost a significant relationship, job or educational or career opportunity because of gambling.

Trichotillomania, pathological hair-pulling, is defined as ‘recurrent failure to resist impulses to pull out one's hair, resulting in noticeable hair loss’. It is a common disorder characterized by plucking of hairs from head, eyelashes, eyebrows and other parts of the body. Trichotillomania is an often chronic and socially debilitating disorder.

From the DSM-IV: Impulse Control Disorders Not Otherwise Specified (NOS):

Compulsive buying disorder, also referred to as compulsive shopping disorder, or uncontrolled buying, is generally recognized as an impulse control disorder. It shares many characteristics with kleptomania.

Binge eating disorder (BED) is characterized by discrete periods of binge eating during which large amounts of food are consumed in a discrete period of time. A sense of control over eating is absent. Binge eating disorder is distinguished from Bulimia Nervosa by the absence of the regular use of inappropriate compensatory behaviors such as self-induced vomiting, misuse of laxatives and other medications, fasting and excessive exercise that are characteristic of the latter. Binge drinking disorder is—mutatis mutandis—the same as binge eating disorder.

Impulsive self injurious behavior (also referred to as repetitive self-mutilation), is the inability of an individual to control the impulse to scratch, pick, lick or cause self-injury by repeated mechanical irritation of an injured area. This behavior becomes manifest in different forms, for instance pathological skin picking (PSP), a severe and chronic psychiatric and dermatologic problem which can lead to significant suffering, dysfunction and disfigurement e.g. in the form of facial skin lesions; pathological nail-biting (onychophagia); pathological nose-picking (rhinotillexo-mania); gouging (auto-enucleation): the use of one's fingers to stick out one's own eyes, a rare and severe form of orbital trauma; head banging and self-biting.

Paraphilic sexual addictions (erotomania, hypersexuality), lack of control of a person's sexual impulses, are characterized by recurrent, intense sexual urges, fantasies, or behaviors that involve unusual objects, activities, or situations and cause clinically significant distress or impairment in social, occupational, or other important areas of functioning. Paraphilias include exhibitionism (exposure of genitals), fetishism (use of non-living objects), frotteurism (touching and rubbing against a nonconsenting person), pedophilia (focus on prepubescent children), sexual masochism (receiving humiliation or suffering), sexual sadism (inflicting humiliation or suffering), transvestic fetishism (cross dressing) and voyeurism (observing sexual activity).

Spending many hours a day behind a computer screen, searching the Internet, may be the characteristic of a well paid job, but it may also cause clinically significant distress or impairment in social, occupational, or other important areas of functioning, in particular when it is not job-related, and restricted to a person's leisure time. Already some clinicians have listed ‘compulsive Internet use’ as impulse control disorder.

In the eyes of many adults, most teenagers make excessive use of mobile phones. Most juveniles perceive this as quite normal. In certain individuals however, this behavior reaches pathological heights. It therefore may be anticipated that ‘excessive mobile phone use’ will be recognized as impulse control disorder, and very well may prove not to be refractory to pharmacological treatment.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and the claims of this specification, the word “comprise” and variations of the word, such as “comprising” and “comprises” is not intended to exclude other additives, components, integers or steps.

The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

From the binding affinity measured for a muscarinic agonist, one can estimate a theoretical lowest effective dose. At a concentration of the compound equal to twice the measured Ki-value, nearly 100% of the muscarinic receptors likely will be occupied by the compound. By converting that concentration to mg of compound per kg of patient one obtains a theoretical lowest effective dose, assuming ideal bioavailability. Pharmacokinetic, pharmaco-dynamic, and other considerations may alter the dose actually administered to a higher or lower value. The dose of the compound to be administered will depend on the relevant indication, the age, weight and sex of the patient and may be determined by a physician. The dosage will preferably be in the range of from 0.01 mg/kg to 10 mg/kg. The typical daily dose of the active ingredients varies within a wide range and will depend on various factors such as the relevant indication, the route of administration, the age, weight and sex of the patient and may be determined by a physician. In general, oral and parenteral dosages will be in the range of 0.1 to 1,000 mg per day of total active ingredients.

The term “therapeutically effective amount” as used herein refers to an amount of a therapeutic agent to treat or prevent a condition treatable by administrating a composition of the invention. That amount is the amount sufficient to exhibit a detectable therapeutic, preventative or ameliorative response in a tissue system, animal or human. The effect may include, for example, treating or preventing the conditions listed herein. The precise effective amount for a subject will depend upon the subject's size and health, the nature and extent of the condition being treated, recommendations of the treating physician (researcher, veterinarian, medical doctor or other clinician), and the therapeutics, or combination of therapeutics, selected for administration. Thus, it is not useful to specify an exact effective amount in advance. The term “treatment” as used herein refers to any treatment of a mammalian, preferably human condition or disease, and includes: (1) preventing the disease or condition from occurring in a subject predisposed to the disease, but not yet diagnosed as having it, (2) inhibiting the disease or condition, i.e., arresting its development, (3) relieving the disease or condition, i.e., causing the condition to regress, or (4) stopping the symptoms of the disease. As used herein, the term “medical therapy” intends to include prophylactic, diagnostic and therapeutic regimens carried out in vivo or ex vivo on humans or other mammals. The term “subject” as used herein, refers to an animal, preferably a mammal, most preferably a human, who has been the object of treatment, observation or experiment.

### EXAMPLE 1

### Formulations Used in Animal Studies

For subcutaneous (s.c.) administration: to the desired quantity (0.05 mg/ml) of MK801 in a glass tube, some glass beads were added and the substance was milled by vortexing for 20 sec. After addition of 2 ml of solvent (Saline 0.9%), the compound was dissolved by vortexing for 20 sec. The rest of the solvent minus 1 ml was added to the solution and vortexed for 20 sec. Then pH was measured (pH between 5-8) and vortexed (20 sec). The last volume was added to the solution, vortexed for 20 sec and a final pH check was done, the actual pH was noted.

For intraperitoneal (i.p.) administration: to the desired quantity (5 mg/ml) of desmethylclozapine in a glass tube, some glass beads were added and the substance was milled by vortexing for 20 sec. After addition of 2 ml of 1% methylcellulose and 5% mannitol in water, the compound was suspended by vortexing for 20 sec. The rest of the solvent minus 1 ml was added to the suspension and vortexed for 20 sec. Then pH was measured and set with 1 drop of 1.0 M NaOH (pH between 5-8) and vortexed (20 sec). The last volume was added to the suspension, vortexed for 20 sec and a final pH check was done, the actual pH was noted.

For intraperitoneal (i.p.) administration: to the desired quantity (5 mg/ml) of xanomeline in a glass tube, some glass beads were added and the substance was milled by vortexing for 20 sec. After addition of 2 ml of 1% methylcellulose and 5% mannitol in water, the compound was suspended by vortexing for 20 sec. and the tube was placed in an ultrasonic bath for 15 min. Before the rest of the solvent was added the suspension was vortexed for 20 sec. The rest of the solvent minus 1 ml was added to the suspension and vortexed for 20 sec. Then pH was measured and set with 5 drops of 1.0 M NaOH (pH between 5-8) and vortexed (20 sec). The last volume was added to the suspension, vortexed for 20 sec and a final pH check was done, the actual pH was noted.

### EXAMPLE 2

### The 5-Choice Serial Reaction Time Task Protocol

Animals

Male Wistar rats weighing 330-450 g (Harlan, The Netherlands) were housed in pairs under a 12-h light/dark cycle (lights off at 7:00 a.m.) and had daily access to a limited amount of food that kept their body weight at approximately 85% of the free feeding weight. Water was available ad libitum. All procedures were in compliance with the European Communities Council Directive of Nov. 24, 1986 (861609 EEC).

Apparatus

Standard 5-CSRT boxes from MED associates Inc. Georgia, Vt. US were used. The curved wall of the chamber contained five 1.6 cm2 holes, 2.2 cm deep, 2 cm above floor level. Each hole had an infra-red photocell beam crossing the entrance and illuminating a photo-electric cell for head entry detection. In every hole a yellow stimulus light was presented. A food dispenser was located at the opposite wall. The box was illuminated by a 28 Volt 100 mA house light. The 5-CSRT boxes were placed in sound attenuating cubicles. On-line control of the apparatus and data collection were performed by a MED-PC experimental apparatus programming system (Tatham, 1989), running on a Dell Optiplex GX1 PC.

Training

Rats were first trained to collect reinforcement pellets from the food dispenser and to collect reinforcements that were placed in the 5 holes in order to habituate the animals to the 5-CSRT task apparatus. Next, training of the 5-CSRT task started. These sessions started by illumination of the light in one of the holes for 30 seconds (stimulus duration). A response by the rat into the illuminated hole or a response in that particular hole for a short period of time after the illumination (the limited hold 60 sec) was rewarded with the delivery of a food pellet, termination of the trial and a correct response was recorded. The light stimulus was presented in each of the holes 20 times during a session (total of 100 presentations), and the order of presentations was randomized. A response in any other hole (fault response) or a failure to respond at all during the stimulus presentation and the limited hold (omission) resulted in a 5 sec. period of darkness (time out) and termination of the trial. After termination of the trial a variable inter-trial interval was started (1, 3, 5, 7 or 9 sec) after which the next trial was started. Upon reaching a 75% correct performance level the duration of illumination of the light stimulus was reduced until a presentation of 0.5 sec (from 30 to 20, 10, 5, 2, 1, 0.9; 0.8; 0.7; 0.6; 0.5.) and a limited hold of 5 sec (in three steps from 30 to 20, 10 and 5 sec), was reached. (Robbins, 1993).

The following parameters were measured (Muir, 1996):

Accuracy: the percentage correct responses divided by the total responses made.

Errors of omission: the number of missed stimulus presentations.

Correct responses: the number of correct responses.

Fault responses: the number of responses in the wrong hole.

Anticipatory responses: the number of premature responses made preceding the presentation of the light stimulus.

Persevering responses: responses emitted after an incorrect or correct response.

### EXAMPLE 3

### Activity in the 5-Choice Serial Reaction Time Task

MK801 was tested in a latin hexagon design, meaning that each rat received all test dosages (0, 0.01, 0.025 and 0.05 mg/kg s.c.) in a random order. The results of two different series of experiments are given in table 1:

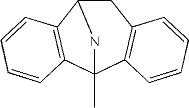

MK-801 (dizocilpine), a non competitive NMDA receptor antagonist

TABLE 1
RESPONSES IN THE 5-CHOICE SERIAL REACTION TIME TASK

| dose of MK-801 (mg/kg, s.c.) | | | |
|---|---|---|---|
| 0 | 0.01 | 0.025 | 0.05 |

First series: numbers (%) of responses (±S.E.M.) at different dosages of
MK801

| | | | | |
|---|---|---|---|---|
| percentage of correct responses | 72.8 ± 3.6 | 72.6 ± 2.8 | 70.0 ± 3.7 | 68.0 ± 3 |
| number of correct responses | 49.8 ± 3.2 | 52.6 ± 3.3 | 53.8 ± 3.9 | 55.3 ± 3.2 |
| number of fault responses | 18.8 ± 2.6 | 19.8 ± 2.2 | 22.6 ± 2.7 | 26.0 ± 2.7 |
| number of missed responses | 31.3 ± 3.1 | 27.7 ± 3.3 | 23.6 ± 2.9 | 18.7 ± 3.1* |
| number of anticipatory | 42.5 ± 6.8 | 51.3 ± 5.4 | 73.2 ± 8.7* | 148.6 ± 38.6* |
| responses | | | | |
| persevering responses | 5.2 ± 0.9 | 7.9 ± 1 | 17.7 ± 4.1* | 28.2 ± 4.7* |
| total number of responses | 68.7 ± 3.1 | 72.3 ± 3.3 | 76.4 ± 2.9 | 81.3 ± 3.1* |

Second series: numbers (%) of responses (±S.E.M.) at different dosages of
MK801

| | | | | |
|---|---|---|---|---|
| percentage of correct responses | 73.4 ± 2.2 | 73.8 ± 2.3 | 74.0 ± 2.5 | 72.7 ± 2.6 |
| number of correct responses | 56.0 ± 3.3 | 60.8 ± 2.1 | 62.7 ± 2.9 | 62.3 ± 2.9 |
| number of fault responses | 19.8 ± 1.4 | 21.8 ± 2.1 | 21.8 ± 1.9 | 23.3 ± 2.3 |
| number of missed responses | 24.3 ± 2.8 | 17.5 ± 2.1 | 15.6 ± 1.9* | 14.5 ± 1.8* |
| number of anticipatory | 67.5 ± 13.1 | 78.1 ± 14.8 | 97.3 ± 16.2 | 148.3 ± 29.8* |
| responses | | | | |
| persevering responses | 6.9 ± 2.0 | 11.1 ± 3.1 | 10.7 ± 1.7 | 18.8 ± 4.2* |
| total number of responses | 75.8 ± 2.7 | 82.5 ± 2.1 | 84.4 ± 1.9* | 85.5 ± 1.8* |

From the data in table 1 it is evident that MK801 does not influence the numbers of correct responses and fault responses. The significant effects are on the number of anticipatory responses, persevering responses, missed responses and total number of responses: those were boosted except for missed responses which were decreased, an effect evident at 0.025 mg/kg and 0.05 mg/kg, and highly significant (*=p<0.05). This indicates that the animals showed impulsive behavior.

Xanomeline, a mixed muscarine-M₁ and muscarine-M₄ agonist, and desmethylclozapine, a muscarinic-M₁ agonist and muscarinic-M₄ antagonist, were each tested in a latin hexagon design, meaning that each rat received all test-dosages (0, 1, 3 and 10 mg/kg i.p.) in a random order. The results are given in table 2:

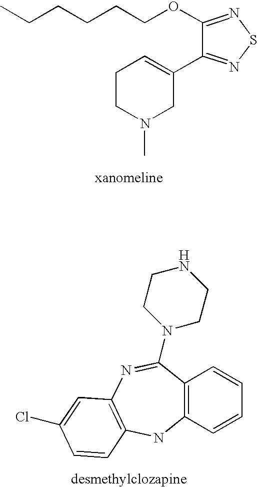

TABLE 2
RESPONSES IN THE 5-CHOICE SERIAL REACTION TIME TASK
Numbers (%) of responses (±S.E.M.)
at different dosages of Xanomeline

| | dose of xanomeline | | | |
|---|---|---|---|---|
| | 0 mg/kg | 1 mg/kg | 3 mg/kg | 10 mg/kg |
| percentage of correct | 70.8 ± 2.3 | 70.0 ± 2.1 | 70.2 ± 2.5 | 72.1 ± 2.3 |
| responses | | | | |
| number of correct | 54.2 ± 2.5 | 53.0 ± 2.4 | 51.2 ± 2.5 | 37.2 ± 2.6* |
| responses | | | | |
| number of fault | 22.2 ± 1.8 | 22.7 ± 1.6 | 21.7 ± 1.8 | 15.0 ± 1.8* |
| responses | | | | |
| number of missed | 23.6 ± 2.1 | 24.3 ± 2.4 | 27.2 ± 2.2 | 46.8 ± 3.7* |
| responses | | | | |
| number of anticipatory | 60.7 ± 7.9 | 58.0 ± 7.9 | 60.8 ± 4.9 | 21.5 ± 6.4* |
| responses | | | | |
| persevering responses | 7.8 ± 1.3 | 6.3 ± 1.1 | 6.7 ± 1.4 | 4.9 ± 0.9 |
| total number of | 76.4 ± 2.1 | 75.7 ± 2.4 | 72.8 ± 2.2 | 53.2 ± 3.7* |
| responses | | | | |

Numbers (%) of responses (±S.E.M.) at different dosages of
Desmethylclozapine

| | dose of desmethylclozapine | | | |
|---|---|---|---|---|
| | 0 mg/kg | 1 mg/kg | 3 mg/kg | 10 mg/kg |
| percentage of correct | 72.6 ± 2.7 | 72.9 ± 3.1 | 71.9 ± 3.2 | 72.6 ± 3.6 |
| responses | | | | |
| number of correct | 51.8 ± 2.7 | 52.2 ± 3.2 | 51.9 ± 3.1 | 48.5 ± 3.9 |
| responses | | | | |
| number of fault | 19.4 ± 2.1 | 19.4 ± 2.2 | 20.1 ± 2.2 | 17.5 ± 2.0 |
| responses | | | | |
| number of missed | 28.8 ± 2.4 | 28.4 ± 3.2 | 28.0 ± 2.2 | 34.0 ± 2.9 |
| responses | | | | |
| number of anticipatory | 40.3 ± 4.2 | 50.2 ± 4.9 | 51.3 ± 7.5 | 34.5 ± 3.9* |
| responses | | | | |
| persevering responses | 7.6 ± 2.1 | 7.8 ± 1.4 | 8.8 ± 1.2 | 5.5 ± 0.9 |
| total number of | 71.2 ± 2.4 | 71.6 ± 3.2 | 72.0 ± 2.2 | 66.0 ± 2.9 |
| responses | | | | |

The data given in table 2 show that at 10 mg/kg xanomeline and desmethylclozapine reduced the number of anticipatory responses: An effect associated with impulsive behavior (Cole, 1987; Ruotsalainen, 2000).

### Interaction Studies

Both xanomeline and desmethylclozapine were each tested in the same dose range against 0.05 mg/kg (s.c.) MK801, a dose shown to elicit unequivocal impulsive behavior (see Table 1). The results are given in table 3:

TABLE 3
RESPONSES IN THE 5-CHOICE SERIAL REACTION TIME TASK
Numbers (%) of responses (±SEM) at different dosages of xanomeline against MK801

| | dose of xanomeline | | | | |
|---|---|---|---|---|---|
| | placebo | 0 mg/kg | 1 mg/kg | 3 mg/kg | 10 mg/kg |
| % correct resp | 71.2 ± 2.6 | 66.9 ± 3.5 | 70.2 ± 3.8 | 71.6 ± 2.4 | 74.4 ± 2.8 |
| nr of correct resp | 51.1 ± 3.2 | 53.3 ± 3.9 | 58.7 ± 4.4 | 57.4 ± 2.7 | 50.9 ± 2.4 |
| nr of fault resp | 20.2 ± 1.6 | 25.9 ± 2.7 | 23.8 ± 2.4 | 22.7 ± 1.9 | 18.0 ± 2.2 |
| nr of missed resp | 28.8 ± 3.0 | 20.8 ± 3.4 | 17.6 ± 3.1 | 19.9 ± 2.4 | 31.1 ± 3.0 |
| nr anticipatory resp | 40.7 ± 3.8 | 133.8 ± 23.5 | 75.3 ± 10.5 | 99.3 ± 11.1 | 47.5 ± 8.4* |
| persevering resp | 6.5 ± 1.7 | 16.9 ± 2.6 | 15.3 ± 2.5 | 14.1 ± 3.5 | 8.5 ± 1.1 |
| total nr of resp | 71.3 ± 3.0 | 79.3 ± 3.4 | 82.4 ± 3.1 | 80.1 ± 2.4 | 68.9 ± 3.0 |

Numbers (%) of responses (±SEM) at different doses of desmethylclozapine vs MK801

| | desmethylclozapine | | | | |
|---|---|---|---|---|---|
| | placebo | 0 mg/kg | 1 mg/kg | 3 mg/kg | 10 mg/kg |
| % correct | 74.3 ± 3.3 | 66.2 ± 3.8 | 67.2 ± 2.6 | 69.6 ± 2.6 | 70.1 ± 3.1 |
| responses | | | | | |
| nr of correct resp | 52.8 ± 3.1 | 53.8 ± 4.4 | 52.5 ± 3.4 | 55.2 ± 2.8 | 52.7 ± 3.9 |
| nr of fault resp | 18.2 ± 2.3 | 25.9 ± 2.1 | 25.1 ± 1.9 | 24.4 ± 2.4 | 21.6 ± 2.0 |
| nr of missed resp | 29.0 ± 2.7 | 20.3 ± 3.7 | 22.4 ± 3.1 | 20.4 ± 3.6 | 25.8 ± 3.7 |
| nr anticipatory resp | 46.9 ± 4.9 | 116.8 ± 19.9 | 141.8 ± 20.8 | 104.3 ± 18.4 | 78.6 ± 14.5* |
| persevering resp | 6.3 ± 1.4 | 20.2 ± 3.2 | 24.8 ± 2.8 | 16.3 ± 2.1 | 17.3 ± 3.4 |
| total nr of resp | 71.0 ± 2.7 | 79.1 ± 3.7 | 77.6 ± 3.1 | 79.6 ± 3.6 | 74.3 ± 3.7 |

The data given in table 3 clearly indicate that both xanomeline and desmethylclozapine antagonize the increase in anticipatory and persevering responses induced by MK801

We claim:

1. A method for treating, ameliorating, or preventing at least one impulse control disorder comprising administering a pharmaceutical composition comprising at least one muscarinic agonist to a patient suffering from at least one impulse control disorder.

2. The method as claimed in claim 1, wherein said muscarinic agonist has muscarinic-1 (M₁) agonistic activity.

3. The method as claimed in claim 2, wherein said muscarinic agonist is chosen from AF-150, AF-151, alvameline, ACP-104, CDD-34, CDD-98, CDD-0097, CDD-0102, CDD-190, CDD-0199-J, CDD-0235-J, CDD-0304, cevimeline, CPR-2006, CS-932, desmethylclozapine, FPL-14995, FPL-15467, KAD-193R, L-680648, L-687306, L-689660, MCNa-343, milameline, nebracetam, NGX-267, PD-151832, sabcomeline, SDZ-210-086, SR-46559A, talsaclidine fumarate, tazomeline, xanomeline, YM-796 and YM-954.

4. The method as claimed in claim 3, wherein said muscarinic agonist is xanomeline.

5. The method as claimed in claim 3, wherein the muscarinic agonist is desmethylclozapine.

6. The method as claimed in claim 1, wherein said impulse control disorder is an impulse control disorder Not Elsewhere Classified.

7. The method as claimed in claim 5, wherein said impulse control disorder—Not Elsewhere Classified is chosen from intermittent explosive disorder, pyromania, kleptomania, pathological gambling and trichotillomania.

8. The method as claimed in claim 1, wherein said impulse control disorder is an impulse control disorder Not Otherwise Specified.

9. The method as claimed in claim 7, wherein said impulse control disorder Not Otherwise Specified is chosen from compulsive buying disorder, binge eating disorder, binge drinking disorder, impulsive self-injurious behavior, paraphilic sexual addictions, compulsive Internet use, and excessive mobile phone use.

10. The method as claimed in claim 9, wherein the self-injurious disorder is chosen from, pathological skin picking, nail-biting and nose-picking, gouging, head banging and self-biting.

11. The method as claimed in claim 9, wherein the paraphilic sexual addiction is chosen from exhibitionism, fetishism, frotteurism, pedophilia, masochism, sadism, transvestic fetishism and voyeurism.
